(19) United States
(12) Patent Application Publication
(10) Pub. No.: US 2012/0070879 A1
(43) Pub. Date: Mar. 22, 2012

(54) REDUCTION OF ANTIBIOTIC ACTIVITY OR CONCENTRATION IN BIOLOGICAL SAMPLES USING MOLECULARLY IMPRINTED POLYMERS

(75) Inventor: Robert Sallitt, Cockeysville, MD (US)

(73) Assignee: BECTON, DICKINSON AND COMPANY, Franklin Lakes, NJ (US)

(21) Appl. No.: 13/321,320

(22) PCT Filed: Jun. 7, 2010
(86) PCT No.: PCT/US10/37594

§ 371 Date: Nov. 18, 2011

Related U.S. Application Data
(60) Provisional application No. 61/184,913, filed on Jun. 8, 2009.

(51) Int. Cl. C12N 1/20; C08G 77/04; B01L 3/00; C12M 1/24; C12M 1/00; C08F 2/02; B01D 15/08

(52) U.S. Cl. 435/252.1; 526/205; 526/204; 528/21; 210/690; 210/692; 210/679; 435/304.1; 435/289.1; 422/549; 422/557

(57) ABSTRACT

Methods of removing undesired compounds such as antibiotics from a biological sample are provided. The methods use Molecularly Imprinted Polymers (“MIPs”) to remove or inactivate antibiotics in a sample. The methods use MIPS to remove or inactivate compounds such as antibiotics in a cell culture.

### CROSS REFERENCE TO RELATED APPLICATION

This application claims the benefit of the filing date of United States Provisional Patent Application No. 61/184,913 filed Jun. 8, 2009, the disclosure of which is hereby incorporated herein by reference.

### BACKGROUND OF THE INVENTION

Methods of reducing the concentration of compounds in a sample are described herein. Methods of reducing the concentration and/or activity of antibiotics in a sample using Molecularly Imprinted Polymers (“MIPs”) are described herein. Methods of reducing the concentration and/or activity of antibiotic compounds in a cell culture are described herein.

Biological activity in blood, such as bacteremia (bacteria in blood), remains a significant problem despite the availability of antimicrobial drugs. In particular, the rapid isolation of certain organisms from bacteremic patients is made more difficult when the patient has been administered antibiotics, which are transferred along with the bacteria in the blood into culture broths, since the antibiotics often inhibit the growth of the organism in the culture media. It is nevertheless important that the identification and susceptibility of an infecting organism to various antimicrobial drugs be determined as early as possible in the course of bacteremia and the adverse effects of antibiotics on bacteremia need to be mitigate.

When conventional culturing techniques are employed to identify an infecting organism, the administration of an antibiotic prior to drawing the blood sample for testing can result in inhibition of the growth of bacteria, thus interfering with isolation and identification of the bacteria. Even when antibiotics may not be present in the blood, the isolation of the offending organism(s) still may require excess periods of incubation because of inhibitors contained in serum, plasma, or lysed erythrocytes.

Similar problems exist in other types of bodily fluid being examined, such as, for example, urine, spinal fluid, abscess exudates, serum, peritoneal fluid and the like.

Antibiotics inhibit bacteria through a variety of mechanisms. One of these mechanisms inhibits the bacteria's ability to synthesize its cell wall. Antibiotics belonging to the β-lactam or cephalosporin groups inhibit cell wall synthesis. Examples of cephalosporins include penicillin G, ampicillin, amoxicillin, carbenicillin, nafcillin, ticarcillin, cefamandole, cefotaxime, cefoxitin, cephalexin, cephaloridine, cephalothin and moxalactam.

The bacterial cell wall allows the cell to live in a fluid environment, which is less dense than the interior of the cell. Because the bacterium's cytoplasm (interior) is much denser than the fluid surrounding the cell, there is a large osmotic pressure difference between the two. This causes water to be drawn into the bacterium and, were it not for its rigid cell wall, the cell would swell and burst. When synthesis of the cell wall is inhibited by antibiotics, the cell wall is weakened and osmotic pressure can cause lysis (bursting) thereby killing the bacteria.

Weakening of the bacterium's cell wall by an antibiotic does not take place instantaneously, but usually over a period of hours. The time required is influenced by several factors, including the concentration of antibiotics and the rate of growth of the bacterium. If a patient has a bacterial infection of the blood and is being treated with a β-lactam or cephalosporin class antibiotic, there is a probability that at a given time during treatment there is a population of bacteria in the blood which has been weakened, but not killed by the antibiotic. A sample of this patient's blood, when added to normal blood culture medium, may show no viable bacteria to be present, for cell-wall-damaged bacteria in the blood may lyse due to osmotic stress in the culture medium, which would be a false negative result.

Adding a carbohydrate saccharide, such as sucrose (for example) to a culture medium increases its density. Such media are described as hypertonic. In hypertonic media, there is less of a difference in density between the bacterium's cytoplasm and the surrounding fluid. This causes less of an osmotic differential and therefore less osmotic stress on the bacterial cell. Cell wall-damaged bacteria can thus survive more easily in hypertonic media because the hypertonic solution mitigates the adverse consequences of the antibiotic—weakened bacteria cell wall.

The usual method for detection of bacteria is to inoculate 5 ml of a bodily fluid into a culture medium and wait for the appearance of turbidity which is an indication of bacterial growth. Patients who have been subjected to antibiotic therapy will have the antibiotic present in the bodily fluid at the time the fermentation is initiated. Presence of the antibiotic inhibits growth of the bacteria and may delay isolation of the bacteria for as long as 14 days or longer.

Presently, biological activity is measured using fluorescent detection (BACTEC 9000 series blood culture instruments form Becton Dickinson) and colorimetric detection (BacT/ALERT® 3D Microbial Detection System from bioMérieux). BACTEC 9000 series incubates a sample vial. The BACTEC instrument contains a sensor that monitors the CO₂ produced by metabolism of microorganisms grown in the culture. An increase in fluorescence indicates an increase in the amount of CO₂ or a decreasing amount of O₂ in the sample vial. US Patents that describe aspects of fluorescent detection of the growth of microorganisms include U.S. Pat. Nos. 5,372,936 and 5,266,486 to Fraatz. Similarly, colorimetric detection of microorganisms tracks CO₂ production using a color sensor that changes color when CO₂ levels exceed a certain threshold. Microorganisms so detected include aerobic, anaerobic, and facultative anaerobic microorganisms, as well as yeast and fungi. US Patents that describe aspects of colorimetric detection are U.S. Pat. Nos. 5,217,876, 5,164,796, 5,094,955 and 4,945,060. Although these methods can be used to rapidly determine the presence of bacteria in a culture, challenges are faced when the culture is performed with antibiotics or other inhibitors present in the sample or culture media. Blood culture media can be prepared with the inclusion of antibiotic-neutralizing resins and charcoal. These resins and charcoals bind certain classes of antibiotic molecules that possess a charged functional group or a surface property such as hydrophobicity and that are nonspecifically neutralized. The binding efficiency varies with the type of resin or charcoal selected and usually multiple resins/charcoal must be included in the media to effectively bind broad classes of antibiotics. Also, new antibiotics that do not exhibit typical surface properties will not be bound effectively by these resins/charcoal. Additionally, the presence of resins/charcoal in the media may exhibit undesirable side effects such as sequestering of nutrients required for microbial growth and or producing artifacts that can interfere with proper performance of down-stream testing such as Gram staining.

Antibiotics can also be separated from microorganisms by membrane chromatography, but these procedures are not practical because of the complexity of the separation technique and the high rate of contamination of the test culture.

U.S. Pat. No. 4,174,277 to Melnick et al. describes a method for separation of antibiotics from microorganisms present in a body fluid sample. In the described method, an antibiotic is selectively removed from a bacterially infected body fluid specimen by adsorbing the antibiotic onto a resin system treated with a detergent. The detergent renders the resin system selective for the antibiotic while permitting the bacteria to remain free in the eluting fluid and thus the bacteria are separated from the antibiotic. The eluted body fluid specimen containing the bacteria is then inoculated into a growth medium and cultured. A device and method for collecting blood where affinity molecules are disposed in a reservoir of samples collection tube are described in US Patent Publication No. 2007/0020629 to Ross et al. and entitled “Device for Component Removal During Blood Collection, and Uses Thereof.” The affinity molecules bind an undesired component within the blood and the undesired component is removed from the sample.

Other methods of removing antibiotic and inhibitor contamination of blood samples that rely on using resin or charcoal are described in U.S. Pat. Nos. 4,632,902, 5,162,229, 5,314,815, and 5,624,814.

### BRIEF SUMMARY OF THE INVENTION

Methods and materials for mitigating the adverse effects of the presence of antibiotics in MIPs provide an alternative to using methods based on resin or charcoal. By targeting specific antibiotics with MIPs designed to recognize those antibiotics, one can specifically reduce the activity of antibiotics that are not effectively neutralized with the resin or charcoal approach and avoid adverse side effects or complications associated with those methods.

Described herein are methods for: i) reducing the concentration of an antibiotic (or class of antibiotics) in a sample; ii) removing antibiotics (or a class of antibiotics) from a sample; and iii) reducing the activity of an antibiotic (or class of antibiotics) in a sample. The methods involve adding to a biological sample a MIP that specifically binds to the antibiotic in the sample. The MIP specifically binds the antibiotic in an amount effective to remove antibiotics or reduce the activity or concentration of the antibiotic in the sample.

In one embodiment of the described method the activity of a class of antibiotics in a fluid sample is reduced by adding to the fluid sample a MIP that specifically binds to antibiotics in the class of antibiotics. The MIP specifically binds to at least one antibiotic in the class of antibiotics in an amount effective to reduce the activity of the at least one antibiotic.

In another embodiment of the described method, the concentration of an antibiotic in a fluid sample is reduced by collecting the fluid sample in a collection device that contains MIPs. The MIPs are attached to at least a portion of a surface of the collection device that contacts the fluid sample. The MIPs specifically bind the antibiotic, allowing the antibiotic to specifically bind to the MIPs attached to the collection device. The fluid sample is then removed from the collection device but the MIPs with unwanted antibiotics bound thereto remain, thereby reducing the concentration of the antibiotic in the fluid sample.

In further embodiments, the method described herein reduces the concentration of antibiotics in a fluid sample by collecting the fluid sample in a collection device, wherein MIPs are attached to at least a portion of a surface of the collection device that contacts the fluid sample. The MIPs specifically bind to certain antibiotics in a class of antibiotics. At least one antibiotic in the class of antibiotics specifically binds to the MIPs attached to the collection device. The fluid sample is removed from the collection device with the MIPs with antibiotic bound thereto remaining behind. Because the antibiotic bound to the MIPs remains behind, the concentration of antibiotics in the fluid sample is reduced.

In another embodiment, the method described herein is used to reduce the activity of an antibiotic in a culture. In this embodiment a culture is inoculated with bacterial cells. The cell culture contains media that includes MIPs that specifically bind to the antibiotic. The MIPs specifically bind to the antibiotic, which reduces the activity of the antibiotic.

In another embodiment the method described herein is used to reduce the activity of antibiotics in a culture. According to the method, a culture is inoculated with bacterial cells. The culture contains media that includes MIPs that specifically bind to antibiotics in a class of antibiotics. The MIPs specifically bind to at least one antibiotic in the class of antibiotics in an amount effective to reduce the activity of the at least one antibiotic in the sample.

In yet another embodiment, the method described herein is used to reduce the concentration of an antibiotic in a fluid sample by adding MIPs attached to beads to the fluid sample. The MIPs attached to the beads can specifically bind to the antibiotic. The antibiotic is allowed to bind to the MIPs attached to beads, and the beads are then separated from the fluid. The concentration of the antibiotic in the fluid sample is thereby reduced because the antibiotic remains bound to the MIPs that are separated from the fluid sample.

In yet another embodiment, the method described herein is used to reduce the concentration of antibiotics in a fluid sample by adding MIPs attached to beads to the fluid sample. The MIPs attached to the beads specifically bind to antibiotics in a class of antibiotics. At least one antibiotic in the class of antibiotics is allowed to bind to the MIPs attached to beads. The beads are then separated from the fluid. This reduces the concentration of the at least one antibiotic in the fluid sample.

The method described herein is used to reduce the concentration of an antibiotic or antibiotics from a fluid sample. In this embodiment the fluid sample is collected in a collection device. MIPs are contained within the collection device and contact the fluid sample in the collection device. The MIPs specifically bind the antibiotic or antibiotics in a class of antibiotics. The antibiotic or at least one antibiotic in the class of antibiotics specifically binds to the MIPs contained within the collection device. The fluid sample is removed from the collection device. The concentration of the antibiotic or the at least one antibiotic in the class of antibiotics is reduced in the fluid sample since the antibiotics remain bound to the MIPs that remain in the collection device.

In further embodiments, a kit is provided for culturing cells. The kit contains media that has at least one MIP specific for an antibiotic or class of antibiotics.

In further embodiments, a collection device is provided for the collection of a fluid sample. At least one MIP specific for an antibiotic or class of antibiotics is attached to at least one surface of the collection device. The at least one surface of the collection device is intended to come in contact with the fluid sample.

In further embodiments, a collection device is provided for the collection of a fluid sample. MIPs are contained within the collection device and are intended to contact the fluid sample in the collection device. The MIPs are specific for an antibiotic or class of antibiotics.

The methods described herein are suited for removing antibiotics from or reducing the activity or concentration of antibiotics in any biological sample for any downstream analysis where the presence of antibiotics might otherwise adversely affect the results. In addition to removing antibiotics from or reducing the activity or concentration of antibiotics in blood cultures, the methods are used for treating sample for downstream sample identification testing or antibiotic susceptibility testing.

### BRIEF DESCRIPTION OF TEE DRAWINGS

FIG. 1 illustrates the adsorption rate of ciproflaxicin to a MIP in a clean buffer without growth medium according to one embodiment of the present invention.

FIG. 2 illustrates the removal rate of ciproflaxicin by a MIP in a buffer containing bacterial growth medium according to one embodiment of the present invention.

FIG. 3 illustrates the removal rate of penicillin G by a MIP in clean buffer without growth medium according to one embodiment of the present invention.

FIG. 4 illustrates the removal rate of penicillin G by a MIP in a buffer containing bacterial growth medium according to one embodiment of the present invention.

FIG. 5 illustrates the removal rate of oxacillin by a MIP in a buffer containing bacterial growth medium according to one embodiment of the present invention.

### DETAILED DESCRIPTION

Unless otherwise defined, all technical and scientific terms used herein have the same meaning as commonly understood by one of ordinary skill in the art. Although methods and materials similar or equivalent to those described herein can be used in the practice or testing of the claims, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

In order that the present invention may be more readily understood, certain terms are first defined. Additional definitions are set forth throughout the detailed description. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, delivery, and treatment of patients.

In this application, the use of the singular includes the plural unless specifically stated otherwise. In this application, the use of “or” means “and/or” unless stated otherwise. In the context of a multiple dependent claim, the use of “or” refers back to more than one preceding independent or dependent claim in the alternative only. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Other features and advantages will be apparent from the following detailed description and claims.

The terms “specific binding” or “specifically binds” refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant K_{A} is higher than 10⁶ M⁻¹. If necessary, nonspecific binding can be reduced without substantially affecting specific binding by varying the binding conditions. The appropriate binding conditions, including but not limited to concentration of MIPs, ionic strength of the solution, temperature, time allowed for binding, concentration of a blocking agent (e.g., serum albumin, milk casein), etc., may be optimized by a skilled artisan using routine techniques. In this regard, specific binding is not necessarily exclusive in that a molecule can specifically bind to more than one other molecule.

The term “specific binding agent” refers to a naturally or non-naturally occurring molecule that specifically binds to a target. In certain embodiments, a specific binding agent is an MIP.

The term “isolated” refers to a molecule that is substantially free of its natural environment. For instance, an isolated antibiotic is substantially free of cellular material or other proteins from the cell or tissue source from which it was derived. The term also refers to preparations where the isolated molecule is at least 70-80% (w/w) pure; or at least 80-90% (w/w) pure; or at least 90-95% pure; or at least 95%, 96%, 97%, 98%, 99%, or 100% (w/w) pure.

The term “in combination” in the context of the administration of two agents means that the agents are administered substantially contemporaneously, either simultaneously or sequentially. If given sequentially, at the onset of administration of the second compound, the first of the two compounds is preferably still detectable at effective concentrations at the site of administration.

The term “kit” refers to a combination of reagents and other materials. In certain embodiments, kits can also include, for example but not limited to, apparatus and reagents for sample collection and/or purification, apparatus and reagents for product collection and/or purification, sample tubes, holders, trays, racks, dishes, plates, instructions to the kit user, solutions, buffers or other chemical reagents, suitable samples to be used for standardization, normalization, and/or control samples. In certain embodiments, kits are packaged for convenient storage and safe shipping, for example, in a box having a lid. In certain embodiments, kits include a sample collection device comprising at least one MIP specific for a target. The MIP is attached to at least one surface of the collection device. In certain embodiments, kits comprise media for cell culture wherein the media comprises at least one MIP specific for a target. In certain embodiments, kits include beads attached to one or more MIPs, wherein the one or more MIPs are specific for a target.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The term “bacteria” refers to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. In certain embodiments, bacteria can be gram negative or gram positive. “Gram negative” and “gram positive” refer to staining patterns with the Gram-staining process which is well known in the art. Examples of bacteria include, but are not limited to the following (and all species of the following), Pseudomonas fluorescens, Pseudomonas aeruginosa, Pseudomonas acidovorans, Pseudomonas alcaligenes, Pseudomonas putida, Pseudomonas syringae, Pseudomonas aureofaciens, Pseudomonas fragi, Fusobacterium nucleatum, Treponema denticola, Porphyromonas gingivalis, Moraxella catarrhalis, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudo tuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Pasteurella multocida, Pasteurella haemolytica, Helicobacter pylori, Campylobacter fetus, Campylobacter jejuni, Campylobacter coli, Borrelia burgdorferi, Vibrio cholerae, Vibrio paramaemolyticus, Legionella pneumophila, Listeria monocytogenes, Neisseria gonorrhoeae, Neisseria meningitidis, Gardnerella vaginalis, Bacteroides spp., Clostridium difficile, Mycobacterium tuberculosis, Mycobacterium avium, Mycobacterium intracellulare, Mycrobacterium leprae, Corynebacterium diphtheriae, Corynebacterium ulcerans, Streptococcus spp., Enterococcus spp., Desulfvibrio spp., Actinomyces spp., Erwinia spp., Xanthomonas spp., Xylella spp., Clavibacter spp., Desulfomonas spp., Desulfovibrio spp., Desulfococcus spp., Desulfobacter spp., Desulfobulbus spp., Desulfosarcina spp.,Deslfuromonas spp., Bacillus spp., Streptomyces spp., Clostridium spp., Rhodococcus spp., Thermatoga spp., Sphingomonas spp., Zymomonas spp., Micrococcus spp., Azotobacter spp., Norcardia spp., Brevibacterium spp., Alcaligenes spp., Microbispora spp., Micromonospora spp., Methylobacterium organophilum, Pseudomonas reptilivora, Pseudomonas carragienovora, Pseudomonas dentificans, Corynebacterium spp., Propionibacterium spp., Xanothomonas spp., Methylobacterium spp., Chromobacterium spp., Saccharopolyspora spp., Actinobacillus spp.,Alteromonas spp., Aeronomonas spp., Agrobacterium tumefaciens, Staphylococcus aureus, Staphylococcus epidermidis, Staphylococcus hominis, Staphylococcus haemolyticus, Staphylococcus warneri, Staphylococcus cohnii, Staphylococcus saprophyticus, Staphylococcus capitis, Staphylococcus lugdunensis, Staphlyococcus intermedius, Staphylococcus hyicus, Staphylococcus saccharolyticus and Rhizobium spp.

The term “antibiotic” refers to any natural or synthetic substance intentionally administered to a subject that inhibits the growth of or destroys microorganisms. For example, and not by way of limitation, the antibiotic may inhibit cell wall synthesis, protein synthesis, nucleic acid synthesis, or alter cell membrane function. Examples of antibiotics include amoxycillin, ampicillin, penicillin, clavulanic acid, aztreonam, imipenem, streptomycin, gentamicin, vancomycin, clindamycin, ephalothin, erythromycin, polymyxin, bacitracin, amphotericin, nystatin, rifampicin, teracycline, coxycycline, chloramphenicol and zithromycin. See, e.g., Gilbert et al., eds. The Sanford Guide To Antimicrobial Therapy 2008, 38^{th} Ed. Sperryville: Antimicrobial Therapy; 2008: 216 pp.

The term “class of antibiotics” refers to antibiotics that share significant structural similarity. Classes of antibiotics are known to those of skill in the art. Classes of antibiotics are all classes of antibiotics including, but not limited to, macrolides (e.g., erythromycin), penicillins (e.g., nafcillin), cephalosporins (e.g., cefazolin), carbepenems (e.g., imipenem, aztreonam), other β-lactam antibiotics, β-lactam inhibitors (e.g., sulbactam), oxalines (i.e. linezolid), aminoglycosides (e.g., gentamicin), chloramphenicols, sulfonamides (e.g., sulfamethoxazole), glycopeptides (e.g., vancomycin), quinolones (e.g., ciprofloxacin), tetracyclines (e.g., minocycline), fusidic acid, trimethoprim, metronidazole, polymixins, streptomycins, clindamycin, mupirocin, rifamycins (e.g., rifampin), streptogramins (e.g., quinupristin and dalfopristin) lipoprotein (e.g., daptomycin), polyenes (e.g., amphotericin B), azoles (e.g., fluconazole), and echinocandins (e.g., caspofungin acetate).

The term “anti-microbial molecule” refers to any natural or synthetic molecule, endogenous or exogenous, that inhibits the growth of or destroys microorganisms. Examples of anti-microbial molecules include, but are not limited to, antibiotics, free radicals, nucleophiles, electrophiles, lactoferricins, antibodies, complement, and defensins.

The term “molecularly imprinted polymer” or “MIP” refers to a compound produced by template-induced formation of specific recognition sites in a material where the template directs positioning and orientation of the material's structural components by a self-assembly mechanism.

Beads and methods of separating beads are known in the art. A “bead” refers to any material to which a MIP can be attached. Beads may be of any shape, including, but not limited to, spheres, rods, cubes, and bars. Beads may be made of any substance, including, but not limited to, silica glass and polymers. Beads may be any size. Beads may have a single MIP attached to them, or may have more than one MIP attached to them. In certain embodiments, the beads comprise coated or uncoated particles comprising at least one of magnetic material, paramagnetic material, silica glass, polyacrylamide, polysaccharide, plastic, latex, polystyrene, and other polymeric substances. Methods of separating beads include, but are not limited to, separation based on density, size, electrical or ionic charge, diffusion, heat, flow cytometry, magnetic force, and directed light.

A MIP is generally described as a cast, polymer or mold of a molecule of interest (called the “target” or “template”) where an affinity exists between the target and the MIP such that a specific target can attach to a specific MIP. This affinity can be based on chemical complementarity and/or shape and other physical properties of the MIP, including but not limited to size and monomer arrangement. Functional monomers are selected for chemical complementarity with the template to provide greater affinity for the target. Design and selection of the template and solvent can also affect affinity. While not being limited to a particular definition or structural embodiment, the affinity between the target and MIP can be similar to that which exists between an antibody and antigen, an enzyme and substrate, or a lock and key.

Specific methods of making and using MIPs are known in the art. Examples of publications and patents that describe making and using MIPs include, but are not limited to, Urraca et al., Anal. Chem. 79:4915-23 (2007); Chianella et al., Anal. Chem. 74:1299-93 (2002); and U.S. Pat. Nos. 4,406,792, 4,415,655, 4,532, 232, 4,935,365, 4,960,762, 5,015,576, 5,110,883, 5,208,155, 5,310,648, 5,321,102, 5,372,719, and 6,872,786. Methods of assembling MIPs that bind to antibiotics are described in Urraca et al.

MIPs are made by adding the target to a solution of binding molecules that can be formed, for example, chemically into a polymer. In certain embodiments, the binding molecules have an affinity for the target and form a complex by any known chemical mechanism (e.g. chemical reaction, Vander Waal's forces, etc.) Binding molecules can be monomers commonly known in the art.

MIPs are formed by building a complex comprising a target molecule and associated attached binding molecules that possess the ability to be formed into a polymer. The target molecule serves as a “template” for the binding molecules. In one embodiment, the complex is dissolved in a larger amount of other polymerizable molecules. In another embodiment, the other polymerizable molecules serve to cross-link the polymer so formed. Crosslinking molecules typically have at least two places to bind other molecules to form a three dimensional structure. In one embodiment, the crosslinkers hold the complexing molecules together after the target molecule or “template” is removed.

MIPs are also designed to specifically recognize and specifically bind specific antibiotic molecules. The polymers are prepared using the technique of molecular imprinting such that an antibiotic of interest is used as a template molecule and allowed to interact with appropriately selected monomers forming a template-monomer complex which is copolymerized, followed by template extraction, resulting in a fixed polymer which retains the specific binding site for the original template antibiotic. The prepared polymer can be used to bind the template antibiotic, when brought together in solution or solid phase.

In certain embodiments, forming a MIP includes forming a complex that will survive the polymerization process and leave behind a suitable set of binding sites when the target is removed.

A MIP is formed by providing one or multiple members of a class of target molecules and/or a base structure common to a class of target molecules, such that the MIP is formed with binding sites that are, individually or as a group, suitable for multiple members of the class. The class can be, for example, a structurally related family of anti-microbial molecules. In certain embodiments, the class can be any class of antibiotics, including, but not limited to, quinolones, tetracyclines, β-lactam antibiotics, sulfonamides, aminoglycosides, or macrolides. In some embodiments, the binding sites are additionally suitable for members of the class in general, including members that were not provided during polymerization of the MIP.

In one example, at least two different antibiotics in a class of antibiotics are: 1) present in a sample; and 2) specifically bind to MIPs provided according to the methods described herein. Binding of the at least two different antibiotics to the MIPs results in reduction of the concentration and/or activity of the at least two different antibiotics in the sample separated from the MIPs.

Examples of binding molecules or monomers that can be used for preparing a MIP as described herein include the following: 5,10,15,20 tetraphenyl-2,7,12,17-tertravinyl porphine, 1,6,11,16-tetraphenyl-4,8,13,19-tetravinyl-tetrabenzporphine, methylmethacrylate, other alkyl methacrylates, alkylacrylates, ally or aryl acrylates and methacrylates, cyanoacrylate, styrene, α-methyl styrene, vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy)ethyl methacrylate 1-acetoxy-1,3-butadiene; 2-acetoxy-3 butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; acryloyl chloride; (R)-α-acryloxy-.beta., β′dimethyl-g-butyrolactone; N-acryloxy succinimide N-acryloxytris(hydroxymethyl) aminomethane; N-acryloly chloride; N-acryloyl pyrrolidinone; N-acryloyl-tris (hydroxymethyl)amino methane; 2-amino ethyl methacrylate; N-(3-aminopropyl)methacrylamide; (o, m, or p)-amino-styrene; t-amyl methacrylate; 2-(1-aziridinyl)ethyl methacrylate; 2,2′-azobis-(2-amidinopropane); 2,2′azobisisobutyronitrile; 4,4′-azobis-(4-cyanovaleric acid); 1,l′-azobis-(cyclohexanecarbonitrile); 2,2′-azobis-(2,4dimethylvaleronitrile); 4-benzyloxy-3-methoxystyrene; 2-bromoacrylic acid; 4-bromo-1-butene; 3-bromo-3,3-difluoropropane; 6-bromo-1-hexene; 3-bromo-2methacrylonitrile; 2-(bromomethyl)acrylic acid; 8-bromo-loctene; 5-bromo-1-pentene; cis-1-bromo-1-propene; β-bromostyrene; p-bromostyrene; bromotrifluoro ethylene; (±)-3-buten-2-01; 1,3-butadiene; 1,3-butadiene-1 ,4-dicarboxylic acid 3-butenal diethyl acetal; I-butene; 3-buten-2-ol; 3-butenyl chloroformate; 2-butylacrolein; N-tbutylacrylamide; butyl acrylate; butyl methacrylate; (o,m,p)-bromostyrene; t-butyl acrylate; (R)-carvone; (S)-carvone; (−)-carvyl acetate; cis 3-chloroacrylic acid; 2-chloroacrylonitrile; 2-chloroethyl vinyl ether; 2-chloromethyl-3-trimethylsilyl-1-propene; 3-chloro-1butene; 3-chloro-2-chloromethyl-1-propene; 3-chloro-2methyl propene; 2,2-bis(4-chlorophenyl)-1, 1-dichloroethylene; 3-chloro-l-phenyl-1-propene; m-chlorostyrene; o-chlorostyrene; p-chlorostyrene; 1-cyanovinyl acetate; 1-cyclopropyl-1-(trimethylsiloxy) ethylene; 2,3-dichloro-1-propene; 2,6-dichlorostyrene; 1,3dichloropropene; 2,4-diethyl-2,6-heptadienal; 1,9 decadiene; 1-decene; 1,2-dibromoethylene; 1,1-dichloro-2, 2-difluoroethylene; 1,1-dichloropropene; 2,6 difluorostyrene; dihydrocarveol; (±)-dihydrocarvone; (−)dihydrocarvyl acetate; 3,3-dimethylacrylaldehyde; N,N′dimethylacrylamide; 3,3-dimethylacrylic acid; 3,3-dimethylacryloyl chloride; 2,3-dimethyl-1-butene; 3,3- dimethyl-I-butene; 2-dimethyl aminoethyl methacrylate; 2,4-dimethyl-2,6-heptadien-1-01; 2,4-dimethyl-2,6 heptadienal; 2,5-dimethyl-1,5-hexadiene; 2,4-dimethyl-1,3 pentadiene; 2,2-dimethyl-4-pentenal; 2,4-dimethylstyrene; 2,5-dimethylstyrene; 3,4-dimethylstryene; divinyl benzene; 1,3-divinyltetramethyl disiloxane; 8,13-divinyl-3,7,12,17-tetramethyl-21H-23H-porphine; 8,13-divinyl-3,7,12,17 tetramethyl-21H-23H-propionic acid; 8,13-divinyl-3,7,12, 17-tetramethyl-21H-23H-propionic acid disodium salt; 3,9 divinyl-2,4,8,10-tetraoraspiro[5,5]undecane; divinyl tin dichloride; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethyl-2(hydroxymethyl)-1,3-propanediol trimethacrylate; ethyl methacrylate; ethyl vinyl ether; ethyl vinyl ketone; ethyl vinyl sulfone; (l-ethylvinyl)tributyl tin; m-fluorostyrene; o-fluorostyrene; p-fluorostyrene; glycol methacrylate (hydroxyethyl methacrylate); GA GMA; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; I-hexadecene; 1,5-hexadien-3,4-diol; 1,4 hexadiene; 1,5-hexadien-3-ol; 1,3,5-hexatriene; 5-hexen-l, 2-diol; 5-hexen-l-ol; hydroxypropyl acrylate; 3-hydroxy-3, 7,11-trimethyl-l,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopule-gol; itaconic acid; itaconalyl chloride; lead (II) acrylate; 3,4-dimethylocta-1,6-dien-3-ol ((±)-:linalool); linalyl acetate; p-mentha-1,8-diene; p-mentha 6,8-dien-2-ol; methyleneamino acetonitrile; methacrolein; [3-(methacryloylamino)-propyl]trimethylammonium chloride; methacrylamide; methacrylic acid; methacrylic anhydride; methacrylonitrile; methacryloyl chloride; 2-(methacryloyloxy) ethylaceto acetate; (3-methacryloxypropyl)trimethoxy silane; 2-(methacryloxy) ethyl trimethyl ammonium methylsulfate; 2-methoxy propene (isopropenyl methylether); methyl-2-(bromomethyl) acrylate; 5-methyl-5-hexen-2-one; methyl methacrylate; N,N′-methylene bisacrylamide; 2-methylene glutaronitrite; 2-methylene-l, 3-propanediol; 3-methyl-1, 2-butadiene; 2-methyl-l-butene; 3-methyl-l-butene; 3-methyl-l-buten-l-ol; 2-methyl-l-buten-3-yne; 2-methyl-l,5-heptadiene; 2-methyl-l-heptene; 2-methyl-l-hexene; 3-methyl-l,3pentadiene; 2-methyl-l,4-pentadiene; (±)-3-methyl-lpentene; (±)-4-methyl-l-pentene; (±)-3-methyl-l-penten-3-ol; 2-methyl-l-pentene; alpha.-methyl styrene; t-amethylstyrene; t-β-methylstyrene; 3-methylstyrene; methyl vinyl ether; methyl vinyl ketone; methyl-2 vinyloxirane; 4-methylstyrene; methyl vinyl sulfone; 4-methylS-vinylthiazole; myrcene; t-β-nitrostyrene; 3-nitrostyrene; I-nonadecene; 1,8-nonadiene; l-octadecene; 1,7-octadiene; 7-octene-l,2-diol; l-octene; l-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-l-ol; 4-penten-2-ol; 4-phenyl-l-butene; phenyl vinyl sulfide; phenyl vinyl sulfonate; 2-propene-l-sulfonic acid sodium salt; phenyl vinyl sulfoxide; 1-phenyl-l-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyano ethylene; tetramethyldivinyl siloxane; trans 3-chloroacrylic acid; 2-trifluoromethyl propene; 2-(trifluoromethyl)propenoic acid; 2,4,4′-trimethyl-lpentene; 3,5-bis(trifluoromethyl)s tyrene; 2,3-bis (trimethylsiloxy)-1,3-butadiene; l-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2chloroethyl sulfone; 4-(vinyl benzyl)-2-chloroethyl sulfone; N-(p-vinyl benzyl)-N,N′-dimethyl amine; 4-vinyl biphenyl (4-phenyl styrene); vinyl bromide; 2-vinyl butane; vinyl butyl ether; 9-vinyl carbazole; vinyl carbinol; vinyl cetyl ether; vinyl chloroacetate; vinyl chloroformate; vinyl crotanoate; vinyl cyclohexane; 4-vinyl-l-cyclohexene; 4-vinylcyclohexene dioxide; vinyl cyclopentene; vinyl dimethylchlorosilane; vinyl dimethylethoxysilane; vinyl diphe-nylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinyl imidizole; vinyl iodide; vinyllaurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinyl quinoline; I-vinyl silatrane; vinyl sulfone; vinyl sulfone (divinylsulfone); vinyl sulfonic acid sodium salt; o-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy)silane; vinyl 2-valerate and the like. Such materials are well known to one of ordinary skill in the art. See, e.g., U.S. Pat. No. 6,872,786 to Murray et al., issued Mar. 29, 2005, which is incorporated herein by reference.

In one embodiment of the present invention the MIP is formed from porphyrin containing binding molecules. In these embodiments the amounts of polymerizable porphyrin, other monomers and crosslinking agent(s) can vary broadly. Generally the specific nature/reactivities of the binding molecules (e.g. the polymerizable porphyrin described above), other monomers, and crosslinking agent(s) chosen as well as the specific sensor application and environment in which the polymer/sensor will be ultimately employed. The relative amounts of each reactant can be varied to achieve desired concentrations of the functional moieties of the binding molecules (e.g. the porphyrin moieties of the polymerizable porphyrin) in the polymer support structure.

Polymerizations are generally conducted in bulk solution by the free-radical method. Similar methodology can be applied to surface grafting and particle coating with the polymer, as described in Dahl et al., “Surface Grafting of Functional Polymers to Macroporous Poly Trimethylolpropane Trimethacrylate,” Chemistry of Materials 7, 154-162 (1995) and Plunkett et al., “Molecularly-Imprinted Polymers on Silica: Selective Supports for High Performance Ligand-Exchange Chromatography,” J. Chromatogr. A 708, 19-29 (1995).

While free radical polymerization is preferred, monomers can also be selected that are polymerized cationically or anionically. Polymerization conditions should be selected that do not adversely affect the target. Any UV or thermal free radical initiator known to those skilled in the art for free radical polymerization can be used to initiate this method. Examples of UV and thermal initiators include benzoyl peroxide, acetyl peroxide, lauryl peroxide, azobisisobutyronitrile (AIBN), t-butyl peracetate, cumyl peroxide, t-butyl peroxide, t-butyl hydroperoxide, bis (isopropyl) peroxy-dicarbonate, benzoin methyl ether, 2,2′azobis(2,4-dimethylvaleronitrile), tertiarybutyl peroctoate, phthalic peroxide, diethoxyacetophenone, and tertiarybutyl peroxypivalate, diethoxyacetophenone, 1-hydroxycyclohexyl phenyl ketone, 2,2-dimethyoxy-2phenyl-acetophenone, and phenothiazine, and diisopropylxanthogen disulfide. See, e.g., U.S. Pat. No. 6,872,786 referred to previously herein.

Acrylate-terminated or otherwise unsaturated urethanes, carbonates, and epoxies can also be used to fabricate the MIP. An example of an unsaturated carbonate is allyl diglycol carbonate (CR-39). Unsaturated epoxies include, but are not limited to, glycidyl acrylate, glycidyl methacrylate, allyl glycidyl ether, and 1,2-epoxy-3-allyl propane.

In certain embodiments, the binding molecules and the polymer that results therefrom are inorganic. The binding molecules may be made of oxides or other polymerizable inorganic compounds. The oxides or other polymerizable compounds may be made of at least one network-forming metal or metalloid, such as, for example, boron, silicon, zirconium, zinc, tin, or aluminum. Inorganic polymers may be produced using a sol-gel process in which a colloid of inorganic particles in a solvent is polymerized to form a gel. See, e.g., Gupta et al., Biotechnology Advances 26:533-547 (2008); Lee et al., “Molecular Imprinting by the Surface Sol-Gel Process: Templated Nanoporous Metal Oxide Thin Films for Molecular Recognition,” Self-Organized Nanoscale Materials, pp. 186-220 (2006) (Adachi M, Lockwood D, eds).

In yet another embodiment, the polymer is an organic-inorganic hybrid. The binding molecules may be organometallic compounds such as, for example, metal alkoxides. The binding molecules may be metalloid alkoxides. Examples of metal or metalloid alkoxides include, without limitation, tetrapropyl zirconate, tetraethyl orthotitanate, tetramethyl orthotitanate, tetraethyl orthosilane, tetraethoxyorthosilicate, 3-aminopropyltriethoxysilane, bis-(2-hydroxy-ethyl)-aminopropyltriethoxysilane, methyltriethoxysilane, methyltrimethoxyorthosilane, phenyltrimethylorthosilane, tetramethylorthosilane, vinyltrimethoxysilane, propyltrimethoxysilane, tetra-n-propoxysilane, tetrabutoxysilane, dimethyldimethoxysilane, dimethyldiethoxysilane, phenyltrimethoxysilane, phenyltriethoxysilane, glycidoxypropyltrimethoxysilane, aluminum tert-butoxide, 3-[N, N-bis(9-anthrylmethyl)amino]propyl-triethoxysilane, or generally, compounds of the formula M(OR)ₓ, where M is a network-forming metal or metalloid, the Rₓ groups are hydrocarbons, and x is chosen according to the bonding capacity of M, that is, four for Si, Ti, and Zr, three for Al and B, etc. The R groups may be identical, or one or more may vary from the others. Also contemplated are molecules in which more than one metal or metalloid center are linked by a group such as, for example, a ring, including without limitation, bis-(trimethoxysilylethyl)benzene, and bis-(trimethoxysilylethyl) benzene.

In certain embodiments, the inorganic/organic hybrid polymer can be an organically modified silicate (ormosil), organically modified ceramic (ormocer), and/or polymeric ceramic (polyceram).

Polymerizations can be carried out by a sol-gel process when an alkoxysilane-type of polymerizable compound is used. In this case, the alkoxysilane metal chelating monomer is mixed with tetramethoxysilane or tetraethoxysilane in aqueous solution. The sol-gel condensation can be conducted in acidic or basic conditions using procedures well known to those practiced in the art.

In certain embodiments, a molecularly imprinted inorganic or inorganic/organic hybrid polymer is produced by one of the following approaches, or a combination thereof: i)the self-assembly or non-covalent approach, in which at least one species of template is present during polymerization and interacts with the monomers and/or polymer prior to and during polymerization through non-covalent and/or ionic interactions; ii) the pre-organized or covalent approach, in which the template or a structurally similar molecule (template analog) is linked to precursor by a reversible covalent bond, which is broken after polymerization to allow removal of the template or template analog.

In certain embodiments, residual reactive groups in the polymer and/or in the precursor are eliminated chemically, for example, by using an end-capping reagent such as, for example, chlorotrimethylsilane or hexamethyldisilazane after the MIP is formed. See, e.g., Díaz-García et al., Microchim. Acta 149:19-36 (2005).

A MIP can be prepared in a wide variety of forms ranging from powders to beads to macro structures such as plates, rods, membranes or coatings or other materials. In certain embodiments, a MIP can be prepared as a coating on a collection device.

In certain embodiments, a mixture comprising (1) at least one organic polymerizable compound and (2) at least one inorganic, organometallic, or organometalloid compound is polymerized to give an organic-inorganic hybrid gel. Compounds (1) and (2) can be chosen from appropriate genera and/or species as described above.

In certain embodiments, the binding molecules may comprise compounds of the formula M(OR)ₓ₋₁OR′, in which the Rₓ₋₁ groups are the same, and R′ differs from the R groups and is chosen to impart a desired property to the MIP. Possible desired properties include, without limitation, hydrophilicity, hydrophobicity, acidity, basicity, aromaticity, fluorescence, the ability to form crosslinks and/or polymeric branches or side chains, or a functional group or structure that is recognized by or has affinity for the target. See, e.g., Díaz-García et al., Microchim. Acta 149:19-36 (2005).

Molecular imprinting creates specific recognition sites in materials, such as polymeric organic materials. Known molecular imprinting techniques involve crosslinking the functional monomer or mixture of monomers (i.e. the binding molecules described above). In the present disclosure, the terms “binding molecule(s)” and “monomer” are used interchangeably but monomers are not necessarily binding molecules that contain moieties that bind to template/target. Various crosslinking molecules are well known to those skilled in the art and described below. During molecular imprinting, the template molecule interacts with a complementary portion of the functional monomer, either covalently or by other interactions such as ionic, hydrophobic or hydrogen bonding, so that recognition sites for the template molecule can be provided in the MIP substrate material. The template molecule is then removed from the MIP substrate to leave a “cavity” or recognition site. Thus, a nonspecific molecule can be shaped to the contours of a specific target, and when the target is removed, the shape is maintained to give the MIP a propensity to rebind the target. This process is known as “molecular imprinting” or “templating.”

Crosslinking agents that impart rigidity or structural integrity to the MIP are known to those skilled in the art, and include di-, tri- and tetrafunctional acrylates or methacrylates, divinylbenzene (DVE), alkylene glycol and polyalkylene glycol diacrylates and methacrylates, including ethylene glycol dimethacrylate (EGDMA) and ethylene glycol diacrylate, vinyl or allyl acrylates or methacrylates, divinylbenzene, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bisphenol A or ethoxylated bis-phenol A, methylene or polymethylene bisacrylamide or bismethacrylamide, including hexamethylene bisacrylamide or hexamethylene bismethacrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, I-methy:L-2-isocyanatoethyl methacrylate, 1,I-dimethyl-2-isocyanaotoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, and the like.

The choice of monomer and cross-linking agent will be dictated by the chemical properties (hydrophilicity, chemical stability, degree of cross-linking, ability to graft to other surfaces, interactions with other molecules, etc.) and physical properties (e.g., porosity, morphology, mechanical stability, etc.) properties desired for the polymer. The polymerization procedures and conditions which are used are conventional. As noted above, MIP constituents and conditions for forming the MIP (e.g. template used, solvent used, etc.) are selected based upon the specific use for the MIP.

Solvent, temperature and means of polymerization (e.g. free radical initiation, y-radiation) can be varied in order to obtain polymeric materials of optimal physical or chemical features, for example, porosity, stability, and hydrophilicity. The solvent will also be chosen based on its ability to solubilize all the various components of the reaction mixture. In certain embodiments, a solvent is added to the mixture of binding molecules and crosslinking molecules. In certain such embodiments, the solvent molecules get caught up in the growing polymer and leave gaps and pores in the structure to make the complexes more accessible after the polymer is formed.

The morphology and selectivity of the polymer for binding the target molecule may be improved by altering the solvent, polymerization temperature, and choice of crosslinking agent, as described in Sellergren et al., “Influence of polymer morphology on the ability of imprinted network polymers to resolve enantiomers,” J. Chromatogr. 635:31-40 (1993). In particular, photoinitiation at low temperature should promote high selectivity and strong binding by the polymer of the invention.

After polymerization is complete, and before the template is removed, the crosslinked polymer can be washed and cut into pieces or ground into powder and extensively eluted with the same solvent used for polymerization to remove any unreacted reagents. The polymer is preferably ground in order to maximize the surface area of the polymer and promote access to the polymer by the eluting solvent. The polymer is preferably cryogenically frozen in liquid nitrogen prior to grinding so that the polymer becomes brittle enough to be ground and to prevent degradation of the polymer by the heat of friction generated during grinding.

Removal of the target molecule leaves a macroporous polymer with complementary molecular cavities which as described above, can include, in one embodiment, porphyrin moieties that have specific binding affinity for the target molecule with which the polymer was imprinted. In certain embodiments, the target molecule is removed from the purified polymer under the mildest conditions possible. In certain embodiments, removal of the target molecule may be accomplished by washing with a suitable organic solvent, such as alcohol or by using a stream of air heated to 40 to 60° C. for a few minutes.

After polymerization of the MIPs, a chunk of plastic is obtained. That chunk of plastic is ground up into a powder and the target molecule is removed by washing with a solvent. This technique results in a powder made up of polymers with holes that have a memory for the target molecule. The powder with the holes can be used to recapture the target molecule.

Among patients in clinical settings where bacteremia is suspected and blood samples are drawn for culture and detection of the causative organism, it is usual that a high percentage of patients have received antibiotic therapy prior to the collection of the blood sample. The presence of active antibiotic in the blood can have a negative effect on the recovery of the blood borne organism; i.e., the antibiotic kills or inhibits the division of the organism, resulting in no growth and yields a false negative culture report. In preferred embodiments, the addition of properly designed MIPs to the blood sample at the time of collection or shortly after collection, inhibits the killing action of the antibiotic, allowing microbial division and growth of a bacterial culture to proceed. Some antibiotics are fast acting and therefore require immediate, rapid, inactivation. The sooner the MIPs are added to the culture after collection, the more effective their inhibitory activity is. Preferably, MIPs are added to the sample within one hour from sample collection or culture inoculation.

According to the disclosed methods, MIPs are used to reduce the concentration of, or remove antibiotics or anti-microbial compounds from a sample when placed in contact with the sample.

In cases where more than one antibiotic has been administered to a patient, two or more different MIPs, each specific for a different antibiotic can be used to reduce the concentration and/or activity of multiple antibiotics in a sample. By way of representative example, if a patient receives two or more different antibiotics, two different MIPs can be used to reduce the concentration of, reduce the activity of, neutralize, or remove the two or more different antibiotics.

In another preferred embodiment, a single set of MIPs can be specific for a predetermined or defined class of antibiotics. The MIP's affinity for an entire class of antibiotics can be obtained by selecting a MIP template with a structural or chemical feature shared by all of the individual antibiotics in the predetermined or defined class. Thus, if a patient is administered two or more different antibiotics from the same class, a single type of MIP specific for the antibiotics of that class can be used to reduce the concentration of, reduce the activity of, neutralize, or remove the two or more different antibiotics. By way of example only, a single MIP can be designed to specifically bind β-lactam antibiotics by using a template for forming the MIP that is a shared chemical or structural feature among the β-lactam antibodies. Advantageously, such a MIP has affinity for an entire class of antibiotics. It may also be useful for a MIP to have an affinity for at least 2, 3, 4, 5, 6, 7, 8, 9 or 10 or antibiotics within a class.

The reduction, neutralization and inhibition of antibiotic activity or concentration by the MIPs is effectuated when the target antibiotic binds to the template. This bound antibiotic is not available to kill the organisms in a sample. This is clinically useful because it either removes antibiotics or otherwise mitigates the adverse affects of antibiotics in a biological sample and allows target organisms to grow in culture.

By way of example only, attaching MIPs to the inside of a collection device, such as a collection tube for a blood sample, allows the MIPs to come into contact with the sample as soon as the sample is collected. The MIPS can be attached or immobilized to the collection device by physical or by chemical bonding. The bonds between the MIP and the support surface can be covalent or non-covalent. The MIP may also be admixed into an adhesive compound and cast or sprayed onto the surface of the collection device. Since binding the MIP to the surface uses MIP functional sites that might otherwise bind to target, a balance must be struck between the degree to which eh MIP is bound to the surface and the extent to which the MIP remains capable of binding target.

In certain embodiments, replacing or supplementing media with specifically designed MIPs allows the manufacturer to target the growing number of antibiotics of interest that are not effectively neutralized with the resin/charcoal approach and avoid adverse side effects or complications associated with those methods.

A MIP can specifically bind to antibiotic molecules present in a biological fluid sample (blood and other fluids) and reduce the activity of the antibiotics in the sample. This reduction can allow culturing of microbes contained in the sample, or allow an increased amount or rate of growth of microbes contained in the sample. In certain embodiments, the specific binding of MIPs to antibiotics in a biological fluid effectively neutralizes the antibiotic activity. In certain embodiments, a MIP specifically binds to other target molecules present in a biological fluid sample (blood and other fluids). The specific binding of MIPs to these target molecules in a biological fluid sample reduces or effectively neutralizes the activity of the target molecules in the sample. Examples of biological fluid samples include, but are not limited to, blood, urine, spinal fluid, abscess exudates, serum, peritoneal fluid, semen, vaginal secretions, mucus and the like.

Examples of other target molecules include any anti-microbial or anti-eukaryotic cell molecules. The methods described herein can be used to reduce the concentration of, reduce the activity of, remove, or inactivate molecules in any bacterial or eukaryotic cell culture.

A kit is provided comprising MIPs, which can be added to a biological fluid sample to reduce or neutralize the activity of a target molecule (e.g. antibiotic) in the fluid. In other embodiments, a MIP binds to an antibiotic in a cell culture media and causes a reduction or neutralization of antibiotic activity in the media.

In other embodiments, a kit is provided with media for growing bacteria that includes MIPs specific for at least one, or two or more targets. In certain embodiments, the kit includes media for growing bacteria with MIPs specific for at least one antibiotic. In other embodiments, the kit includes media for growing bacteria with MIPs specific for two or more antibiotics.

In other examples, a MIP is attached to a solid surface, such as the surface of a collection device for a biological fluid sample. Antibiotics present in the biological fluid are specifically bound by the MIPs attached to the solid surface. When the biological fluid sample is removed from the collection device for testing, the MIP bound antibiotics remain bound to the MIPs in the collection device.

The concentration of an antibiotic or antibiotics in a class of antibiotics in a fluid sample is reduced by providing a mechanism to collect the fluid sample in a collection device. MIPs are contained within the collection device and contact the fluid sample in the collection device. The MIPs specifically bind the antibiotic or class of antibiotics. Antibiotics of the class of antibiotics or the antibiotic specifically bind to the MIPs contained within the collection device. The fluid sample is removed from the collection device. This reduces the concentration of the antibiotic or antibiotics in the fluid sample, because the bound antibiotic or antibiotics remain in the collection device. In one example, the MIPs contained within the collection device are placed in a cartridge. In another, the MIPs are in the form of a powder or are bound to beads. In a further example, the fluid sample flows through at least part of the collection device. The collection device has a filter or frit, and the MIPs are contained upstream of the filter or frit. The MIPs collect/retain the antibiotic, and the MIPs are retained by the filter. Thus, the collection of fluid in the device may be transient, i.e., last only as long as it takes for the fluid to flow through the device. Alternatively, the fluid can flow through and/or into one or more parts of the collection device, and the fluid sample is removed after the antibiotic is collected/removed therefrom.

A collection device is provided for the collection of a fluid sample, wherein MIPs are contained within the collection device. The MIPs are intended to contact the fluid sample in the collection device, and the MIPs are specific for an antibiotic or class of antibiotics. The collection device is, for example, a blood collection tube, blood culture bottle, blood collection needle, blood collection set, capillary blood collection device, microcollection tube, urine collection device, or urine collection cup. In certain embodiments, the collection device comprises an evacuated vessel. In certain examples, the collection device includes growth media. Examples of collection devices are described in U.S. Pat. Nos. 4,024,857, 4,409,991, 5,643,202, 6,171,261, 6,508,987, and 6,617,170, which are hereby incorporated by reference.

In one particular embodiment, the kit includes a collection device with at least one solid surface coated with MIPs specific for at least one target (e.g. antibiotics). Optionally, the solid surface is coated with MIPs specific for two or more targets (e.g. antibiotics).

In other examples, a MIP is attached to one or more beads and these beads are introduced into a biological fluid sample. In these examples, MIPs specifically bind the target antibiotics in the biological fluid sample. The antibiotic-bound MIPs are then separated from the biological fluid using the attached beads as the vehicle for separation (e.g. magnetic separation, filtration, etc.).

MIPs can attach to a solid phase, such as beads, that form a MIP-based affinity column. In these embodiments, the MIPs attached to the solid phase are specific for a particular target (e.g. antibiotic). A fluid sample is run through the MIP-based affinity column. The antibiotic that binds specifically to the MIP is retained on the column and the purified fluid sample is then analyzed (e.g. by cell culture).

In a further embodiment, the kit includes beads. The beads are attached to MIPs specific for at least one target (e.g. antibiotic). In alternate embodiments, the beads are attached to MIPs specific for two or more targets (e.g. antibiotics).

The methods, compositions, and devices described in this specification are generally applicable to purifying samples for tissue culture. In certain embodiments, contaminants other than antibiotics can be removed from a sample or have their concentration in the sample reduced by using MIPs. For example and without limitation, contaminants can be removed from a sample taken from a patient prior to culturing the eukaryotic cells of the patient. In certain such embodiments, the contaminant removed from the sample may be a drug or other compound administered to a patient in a hospital.

The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

### Example 1

A MIP prepared with fluoroquinolone as a template was evaluated to determine if it can efficiently adsorb antibiotics under incubation conditions similar to those in the BD BACTEC™ system.

The MIPs were obtained as SupelMIP™ cartridges from Sigma-Aldrich (catalog #53269-U). Each SupelMIP™ cartridge contains 25 mg of MIP made for fluoroquinolone. The MIPs were transferred into vials containing 30 ml of pH 7.2, 10 mM phosphate buffer with 0.85% sodium chloride. The total weight of MIP used in each vial was 250 mg or 300 mg. Ciprofloxacin, a fluoroquinolone antibiotic, was obtained from the pharmacy. A 60 mg/ml stock solution was prepared. A 1.0 ml or 0.25 ml aliquot of stock solution was dispensed into the prepared vials to yield final antibiotic concentrations in each vial of 200 μg/ml or 50 μg/ml. After the stock solution was dispensed, vials were immediately mixed by inversion and shaking for 1 minute to mix the contents thoroughly. A 1 ml of aliquot of liquid sample was immediately removed and designated as a 1 minute time point sample. The vials were continuously incubated at 35° C. for up to 4 hours in a BD BACTEC™ 9240 instrument. Liquid sample aliquots of 1 ml were withdrawn from the vials after 0.5, 1, 2 and 4 hrs of incubation. The concentration of free antibiotic present in the liquid sample was measured by LC-MS or UV-Vis spectrophotometry.

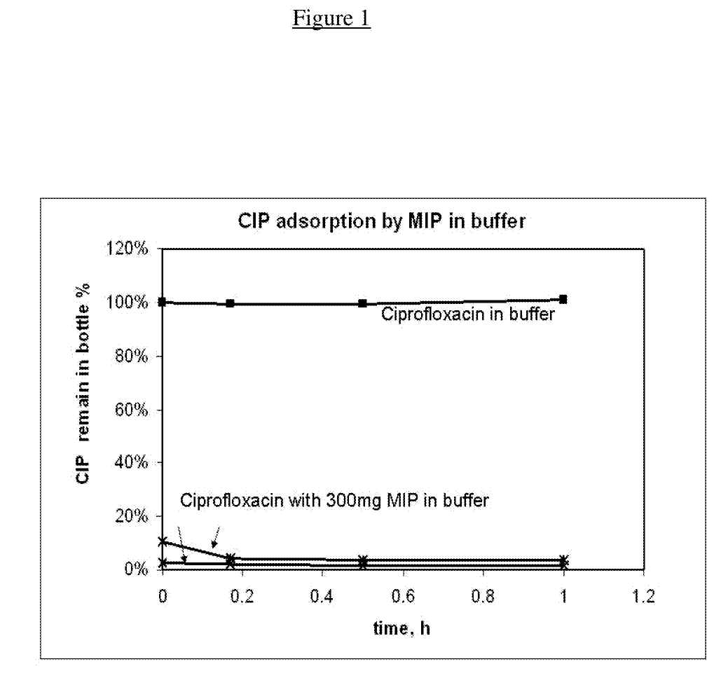

Referring to FIG. 1. The fluoroquinolone-MIP (F-MIP) exhibited rapid adsorption kinetics with ciprofloxacin under the conditions used. More than 95% of ciprofloxacin was bound to 300 mg F-MIP surface in buffer system within 0.5 hour incubation and longer incubation did not improve adsorption.

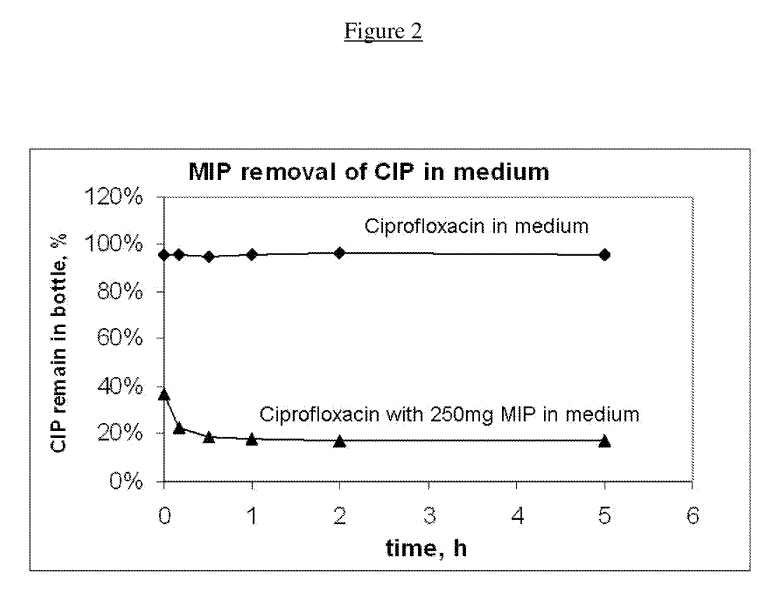

Fast adsorption kinetics with ciprofloxacin by F-MIP was also observed when incubated in the presence of a complex medium system (BD BACTEC™ Standard/10 Aerobic/F Culture Vial, #442260), see FIG. 2. More than 80% of ciprofloxacin was bound to 250 mg MIP surface in bacteria growth medium within 0.5 hour incubation and longer incubation did not improve adsorption. The F-MIP exhibited strong adsorption affinity towards fluoroquinolone despite of multiple ingredients in the medium competing for surface binding sites on MIP. The strong adsorption affinity demonstrates the ability of the methods described to be effective in a complex cellular environment.

To investigate whether F-MIPs are potentially toxic to bacterial growth, a strain of Acinetobacter lwoffii was tested in BD BACTEC™ Plus Aerobic/F medium (catalog #442192) with the presence of 250 mg of F-MIP. The growth of this organism was observed not to be affected by the amount of fluoroquinolone MIP added into the medium. The time to detection of bacterial growth in the vial containing F-MIP was 14.6 hours while time to detection in the vial without F-MIP was 15.5 hours.

### Example 2

A MIP prepared with penicillin G (penG-MIP) as template was evaluated to determine if the MIP can efficiently adsorb β-lactam antibiotic under incubation conditions similar to BD BACTEC™ system.

The penG-MIP were obtained from Dr. Borje Sellergren, University of Dortmund and prepared by the process described in Anal. Chem. 2007, 79, 695-701. The penG-MIP were prepared with penicillin G antibiotic molecule as a template. Prior to use, the penG-MIP were conditioned by washing in series with methanol, methanol-water and acetonitrile, followed by air drying under vacuum and in some cases penG-MIP were used without washing.

Glass vials with screw caps were used and each contained 3 ml of pH 7.2, 10 mM phosphate buffer with 0.85% sodium chloride and penG-MIP. The weight of penG-MIP in each vial was 30 mg or 150 mg.

Antibiotics were obtained from the pharmacy. A stock solution of 6 mg/ml was prepared. A 100 μl or 25 μl aliquot of stock solution was dispensed into the prepared vials to yield final concentration of antibiotics in each vial of 200 μg/ml or 50 μg/ml. After the stock solution was dispensed, vials were immediately capped, inverted and shaken for 1 minute to mix contents thoroughly. A 100 μl aliquot of liquid sample was immediately removed and designated as the 1 minute time point sample. Vials were then placed on a tube rocker (set at ˜20 rpm) and incubated for up to 4 hours in a 35° C. incubator. Aliquots of 100 μl were withdrawn from vials after 0.5, 1, 2 and 4 hours of incubation. All liquid samples withdrawn were dispensed into microcentrifuge spin-filters and spun to remove the residual penG-MIP present in the sample. The concentrations of antibiotics in the liquid samples were then measured using LC-MS or a UV-Vis spectrophotometer with a 50 μL sample cuvette.

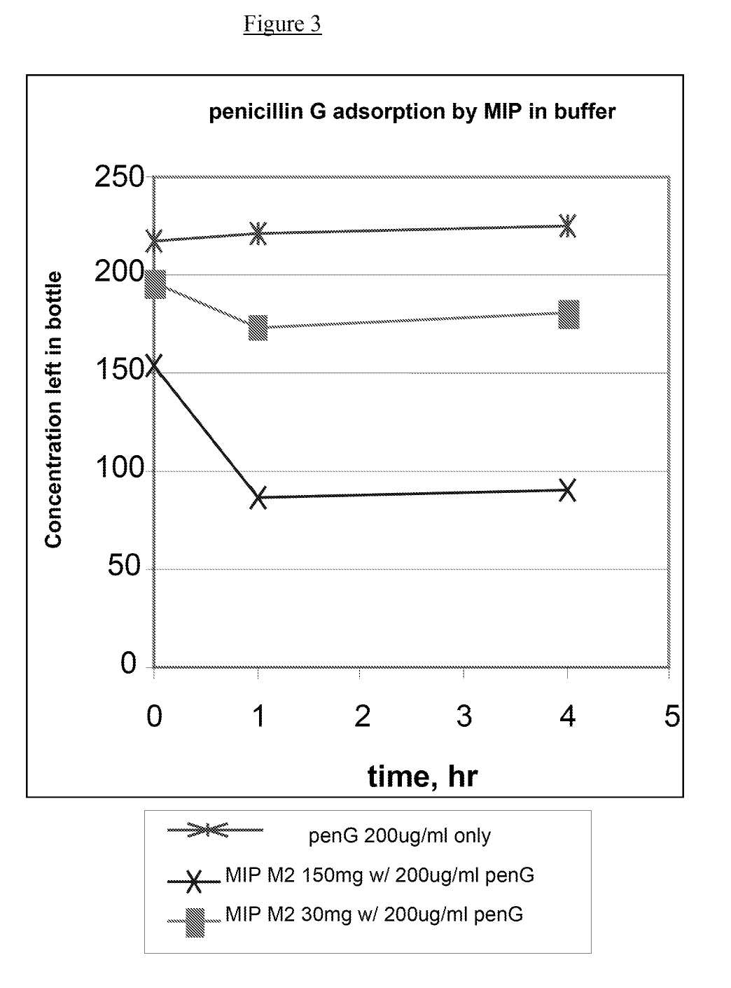

The penG-MIP exhibit rapid adsorption kinetics with penicillin G. Approximately 15% of penicillin G was bound to 30 mg penG-MIP surface after 1 hour incubation and longer incubation did not improve adsorption. Increasing penG-MIP to 150 mg improved adsorption efficiency, resulting in about 50% of the penicillin G being bound to MIP polymer surface. See FIG. 3.

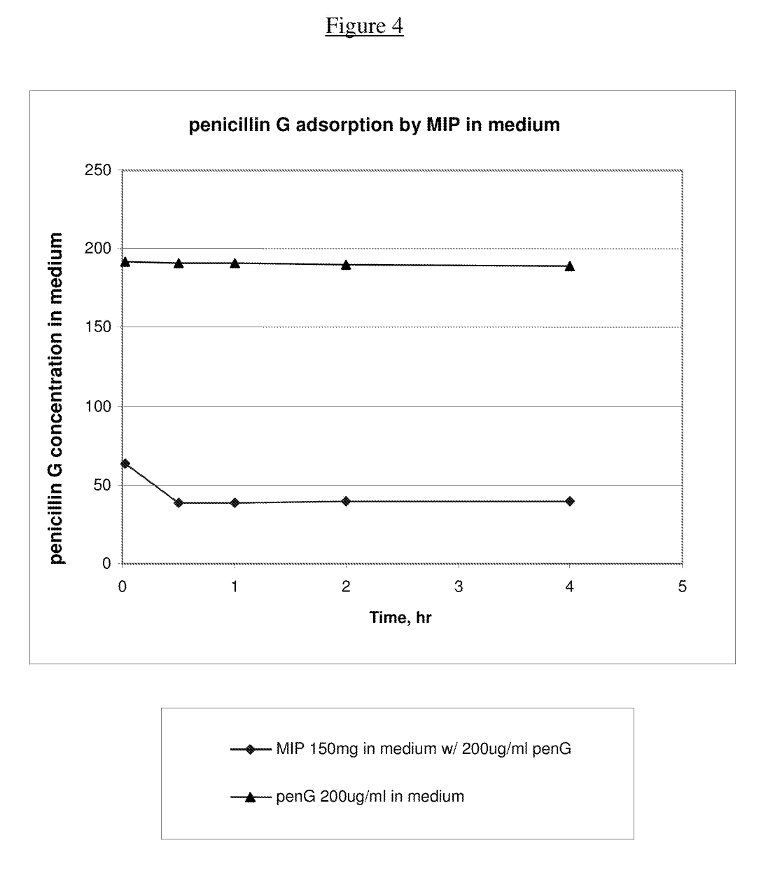

The penG-MIP also exhibited rapid adsorption kinetics with penicillin G in complex growth medium (BD BACTEC™ Standard/10 Aerobic/F Culture Vial, catalog #442260), FIG. 4. Penicillin G adsorption kinetics and efficiency were not affected by multiple competing ingredients in the growth medium. The penG-MIP at a concentration of 150 mg adsorbed more than 50% of penicillin G in the medium after 0.5 hour incubation.

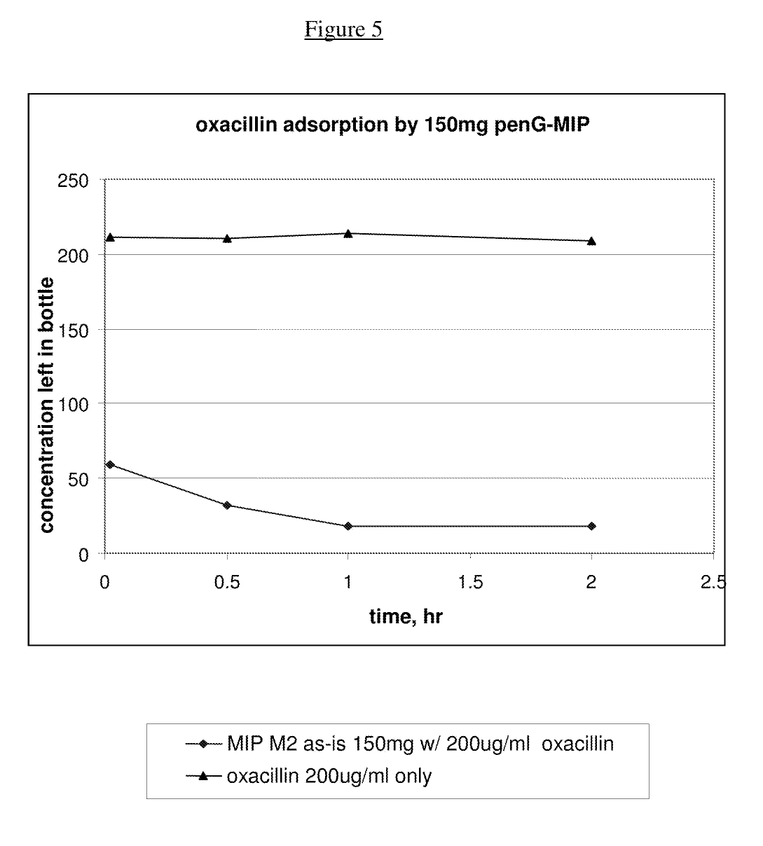

To investigate penG-MIP adsorption towards beta-lactams other than its template penicillin G, oxacillin (which is also a β-lactam with a side chain structure slightly different than penicillin G adsorption was evaluated). As seen in FIG. 5, penG-MIP exhibits rapid kinetics and high efficiency adsorbing oxacillin in a buffer system. About 90% of oxacillin was adsorbed to binding surface of 150 mg MIP within 0.5 hour. PenG-MIP adsorption towards oxacillin was observed to be slightly more efficient than template penicillin G adsorption, illustrating that a MIP may be designed for broad coverage of an antibiotic class by selection of the appropriate template.

1. A method of reducing the activity of an antibiotic in a sample comprising:
adding a Molecularly Imprinted Polymer (MIP) that is configured to bind to the antibiotic in the sample, wherein the MIP binds the antibiotic in an amount effective to reduce the activity of the antibiotic in the sample.

2. The method of claim 1, wherein the sample is a biological fluid sample.

3. The method of claim 1, wherein the sample is blood.

4. A method of reducing the activity of a class of antibiotics in a sample comprising:
adding a Molecularly Imprinted Polymer (MIP) that is configured from a template to bind at least two antibiotics selected from the class of antibiotics in the sample, wherein the MIP binds to the at least two antibiotics in the class of antibiotics in an amount effective to reduce the activity of the at least one antibiotic in the sample.

5. The method of claim 4, wherein the sample is a biological fluid sample.

6. The method of claim 4, wherein the sample is blood.

7. The method of claim 1, wherein the MIP is an organic MIP.

8. The method of claim 1, wherein the MIP is an inorganic MIP.

9. The method of claim 1, wherein the MIP is an organic-inorganic hybrid MIP.

10. A method of reducing the concentration of an antibiotic in a fluid sample comprising:
collecting fluid sample with a first concentration of antibiotic in a collection device, wherein the collection device has Molecularly Imprinted Polymers (MIPs) disposed on at least a portion of a surface of the collection device that contacts the fluid sample, and wherein the MIPs are configured to bind the antibiotic;
contacting the fluid sample with the MIPs, thereby allowing the antibiotic to bind to the MIPs attached to the collection device; and
removing the fluid sample from the collection device, wherein the concentration of the antibiotic in the removed portion of the fluid sample is a second concentration that is less than the first concentration.

11. The method of claim 10, wherein the fluid sample is blood.

12. The method of claim 10 wherein the antibiotic is a class of antibiotics, each with a first concentration in the fluid sample.

13. The method of claim 12, wherein at least two different antibiotics in the class of antibiotics bind to the MIPs, and further wherein the concentrations of the at least two antibiotics in the removed portion of the sample are reduced compared to their respective first concentrations.

14. The method of claim 12, wherein the fluid sample is blood.

15. A method of reducing the activity of an antibiotic in a culture comprising:
inoculating a cell culture with bacterial cells, wherein the cell culture comprises media that further comprises Molecularly Imprinted Polymers (MIPs) that are configured to bind to an antibiotic that may be present in the culture; and
contacting the MIPs with the antibiotic, if present, thereby allowing the MIPs to specifically bind to the antibiotic, thereby reducing the activity of the antibiotic in the cell culture.

16. The method of claim 15, wherein the media comprises the MIPs prior to being combined with the culture.

17. The method of claim 15, wherein the MIPs are added to the media after inoculation of the culture.

18. The method of claim 15 wherein at least two different antibiotics in a class of antibiotics bind the MIPs, and the activity of at least two different antibiotics in the class of antibiotics in the cell culture is reduced by the presence of the MIPs, which are present in the culture in an amount effective to reduce the activity of the at least two antibiotics.

19. The method of claim 18, wherein the media comprises the MIPs prior to being used to prepare the culture.

20. The method of claim 18, wherein the MIPs are added to the cell culture media after inoculation of the culture into the media.

21. A method of reducing the concentration of an antibiotic in a fluid sample comprising:
adding Molecularly Imprinted Polymers (MIPs) attached to beads to the fluid sample, wherein the MIPs attached to the beads are configured to bind to at least one antibiotic;
contacting the MIPs with the fluid sample, thereby allowing the at least one antibiotic to bind to the MIPs attached to beads; and
separating the beads from the fluid sample, thereby removing at least a portion of the antibiotic from the fluid sample and reducing the concentration of the antibiotic in the fluid sample.

22. The method of claim 21, wherein the fluid sample is blood.

23. The method of claim 21, wherein the fluid is a cell culture media.

24. The method of claim 21, wherein the MIPs are configured to bind to at least two different antibiotics in a class of antibiotics, and further wherein the concentrations of the at least two antibiotics are reduced when the MIPs are separated from the fluid sample.

25. The method of claim 21, wherein the fluid is blood.

26. The method of claim 21, wherein the fluid is a cell culture media.

27. A method of reducing the concentration of at least one antibiotic from a fluid sample comprising:
collecting the fluid sample in a collection device, wherein Molecularly Imprinted Polymers (MIPs) are contained within the collection device;
contacting the fluid sample in the collection device with the MIPs, wherein the fluid sample has a first concentration of antibiotics in the sample and wherein the MIPs are configured to bind the at least one antibiotic in a class of antibiotics;
allowing the at least one antibiotic in the class of antibiotics to bind to the MIPs contained within the collection device; and
removing the fluid sample from the collection device, wherein the removed fluid sample has a second concentration of at least one antibiotic in the fluid sample that is less than the first concentration of the antibiotic in the fluid sample.

28. The method of claim 27, wherein the MIPs are configured to bind antibiotics in a class of antibiotics;
wherein the MIPs bind at least two different antibiotics in the class of antibiotics; and wherein the second concentrations of the at least two antibiotics in the removed portion of the fluid sample are less than the first concentrations of the at least two antibiotics in the fluid sample.

29. The method of claim 27, wherein the MIPs contained within the collection device are in a cartridge.

30. The method of claim 27, wherein the MIPs contained within the collection device are in the form of a powder.

31. The method of claim 27, wherein the MIPs contained within the collection device are bound to beads.

32. The method of claim 27, wherein the fluid sample flows through at least part of the collection device, the collection device further comprises a filtration element and configured for the fluid sample to flow through the filter element downstream from where the fluid sample contacts the MIPs, and the MIPs are separated from the fluid sample by the filtration element when the fluid sample flows through the filter element.

33. The method of claim 27, wherein the fluid sample is blood.

34. The method of claim 27, wherein the fluid sample is a cell culture media.

35. The method of claim 10, wherein the MIPs comprise organic MIPs, inorganic MIPS, or organic-inorganic MIPs and combinations thereof.

36. The method of claim 15, wherein the MIPs comprise organic MIPs, inorganic MIPs, or organic-inorganic MIPs and combinations thereof.

37. The method of claim 22, wherein the MIPs comprise organic MIPs, inorganic MIPs, and organic-inorganic hybrid MIPs and combinations thereof.

38. The method of claim 10, wherein the collection device comprises an evacuated vessel.

39. The method of claim 10, wherein the collection device comprises at least one of a blood collection tube, blood culture bottle, capillary blood collection device, microcollection tube, urine collection device, or urine collection cup.

40. The method of claim 10, wherein the collection device comprises growth media.

41. A kit comprising media for culturing cells, wherein the media comprises at least one MIP configured to bind to at least one antibiotic.

42. A kit comprising media for culturing cells, wherein the media comprises at least one MIP configured to bind to a class of antibiotics.

43. A collection device for the collection of a fluid sample, wherein at least one MIP configured to bind to at least one antibiotic is attached to at least one surface of the collection device, and wherein the at least one surface of the collection device is configured to contact fluid sample introduced into the collection device.

44. The collection device of claim 43 wherein at least one MIP is configured to bind at least two antibiotics in a class of antibiotics.

45. The collection device of claim 43 wherein the collection device comprises an evacuated vessel.

46. The collection device of claim 43, wherein the collection device comprises at least one of a blood collection tube, blood culture bottle, capillary blood collection device, blood collection needle, blood collection set, microcollection tube, urine collection device, or urine collection cup.

47. The collection device of claim 45, wherein the collection device comprises growth media.
